(19) United States
(12) Patent Application Publication
(10) Pub. No.: US 2022/0133821 A1
(43) Pub. Date: May 5, 2022

(54) MONITORING AND MODIFYING THE MICROBIOME IN PATIENTS WITH RECTOVAGINAL FISTULA

(71) Applicant: Mayo Foundation for Medical Education and Research, Rochester, MN (US)

(72) Inventors: John A. Occhino, Rochester, MN (US); Marina R. Walther-Antonio, Rochester, MN (US); Douglas Allan Leach, Chesapeake, VA (US)

(21) Appl. No.: 17/519,186

(22) Filed: Nov. 4, 2021

Related U.S. Application Data
(60) Provisional application No. 63/109,638, filed on Nov. 4, 2020.

(51) Int. Cl. A61K 35/748; A23L 33/135

(52) U.S. Cl. CPC A61K 35/748; A23L 33/135

(57) ABSTRACT

Methods and materials involved in assessing and treating a mammal having a rectovaginal fistula (RVF) are provided herein. For example, the methods and materials described herein can be used for determining if a mammal having a RVF is likely to experience a successful outcome after surgical repair of the RVF, or if the mammal is likely to experience recurrence. Methods and materials for modulating the rectal and/or vaginal microbiome of mammal having a RVF also are provided herein.

### CROSS-REFERENCE TO RELATED APPLICATIONS

This application claims benefit of priority from U.S. Provisional Application Ser. No. 63/109,638, filed on Nov. 4, 2020. The disclosure of the prior application is considered part of (and is incorporated by reference in) the disclosure of this application.

### STATEMENT AS TO FEDERALLY SPONSORED RESEARCH

This invention was made with government support under CA136393 and TR002379 awarded by the National Institutes of Health. The government has certain rights in the invention.

### TECHNICAL FIELD

This document relates to methods and materials involved in assessing and/or treating a mammal having a rectovaginal fistula (RVF). For example, methods and materials provided herein can be used to determine if a mammal having a RVF is likely to have a successful outcome after surgical repair of the RVF. This document also provides methods and materials for treating a mammal having a RVF by modulating the rectal and/or vaginal microbiome.

### BACKGROUND

Rectovaginal fistulae are characterized by uncontrolled loss of stool and gas from the vagina as a result of an abnormal communication of the vagina with the rectum. Patients with RVF experience devastating physical and psychosocial effects, including social isolation, sexual dysfunction, vaginitis, cystitis, and life threatening infection. Fistula etiologies include inflammatory bowel disease, infections, radiation therapy, surgical and obstetric trauma (Pinto et al., Dis Colon Rectum 2010, 53(9):1240-1247; and deSouza and Abcarian, “The Management of Rectovaginal Fistula.” In: Current Surgical Therapy, 11th ed., Cameron and Cameron, eds., Philadelphia, Pa., 2014, pp. 283-288). RVF from obstetric trauma are most common and seen primarily in resource poor countries as a consequence of unattended obstructed labor; these account for 2 million fistula worldwide and 50,000 to 100,000 new cases annually (United Nations Population Fund (2015) Obstetric Fistula, from unfpa.org/obstetric-fistula). Rectovaginal fistulae are notoriously difficult to manage, and many require multiple attempts at repair that cumulatively increase the risk of infectious morbidity, perioperative complications, and healthcare costs. Recurrence rates as high as 67% have been reported (Wexner et al., Ann Surg 2008, 248(1):39-43).

### SUMMARY

The microbiome of the mammalian body is involved in maintaining and modulating health and disease, including health and disease in the gastrointestinal and genitourinary systems. RVF involves two distinct microbiome communities, but the potential interplay between these communities has not previously been characterized.

This document is based, at least in part, on the identification of microbiome characteristics associated with fistula recurrence, as well as microbiome characteristics associated with favorable surgical outcomes. As described herein, determination of the rectal and vaginal microbiomes before and after surgical repair of RVF revealed that increased microbiome diversity can be protective from recurrence. Taxa associated with successful repair provide targets for therapeutic intervention, which can increase the likelihood of successful surgical repair.

This document also is based, at least in part, on the development of methods and materials for assessing the likelihood of successful surgical repair in mammals with RVF, as well as materials and methods for increasing the likelihood of successful surgical repair by modulating the rectal and/or vaginal microbiome.

In a first aspect, this document features a method for treating a mammal having a RVF, where the method includes administering to the mammal a composition containing one or more microbes selected from the group consisting of a Cyanobacteria species, a Tenericutes species, a Bacteroidetes species, a Firmicutes species, and a Proteobacteria species. The composition can include one or more microbes selected from the group consisting of a Bacteroidaceae species, a Marinifilaceae species, a Rikenellaceae species, an Acidaminococcaceae species, a Christensenellaceae species, a Ruminococcaceae species, an Alistipes species, a Bacteroides species, a Butyricimonas species, an Odoribacter species, a Paraprevotella species, a Dorea species, a Faecalibacterium species, a Lachnospiraceae species, a Phascolarctobacterium species, a Subdoligranulum species, a Parasutterella species, and a Succinispira species. The mammal can be a human. The method can include administering the composition to the mammal prior to surgical repair of the RVF. The method can further include identifying the mammal as having a rectal or vaginal microbiome with reduced levels of the one or more microbes, as compared to the rectal or vaginal microbiome in a corresponding mammal that does not have a RVF. The identifying can include obtaining a sample from the rectum or vagina of the mammal and determining the rectal or vaginal microbiome within the sample. The composition can contain from about 10³ cfu to about 10¹⁵ cfu of each microbe. The composition can be administered orally, vaginally, or rectally. The composition can be formulated as a tablet, a capsule, a pill, a nutritional supplement, or a beverage. The composition can further include one or more prebiotics. The administering can include fecal microbiota transplantation.

In another aspect, this document features a method for altering the rectal and/or vaginal microbiome in a mammal, where the method includes administering to the mammal a composition containing one or more microbes selected from the group consisting of a Cyanobacteria species, a Tenericutes species, a Bacteroidetes species, a Firmicutes species, and a Proteobacteria species. The composition can include one or more microbes selected from the group consisting of a Bacteroidaceae species, a Marinifilaceae species, a Rikenellaceae species, an Acidaminococcaceae species, a Christensenellaceae species, a Ruminococcaceae species, an Alistipes species, a Bacteroides species, a Butyricimonas species, an Odoribacter species, a Paraprevotella species, a Dorea species, a Faecalibacterium species, a Lachnospiraceae species, a Phascolarctobacterium species, a Subdoligranulum species, a Parasutterella species, and a Succinispira species. The mammal can be a human. The mammal can be identified as having a RVF. The method can include administering the composition to the mammal prior to surgical repair of the RVF. The method can further include identifying the mammal as having a rectal or vaginal microbiome with reduced levels of the one or more microbes, as compared to the rectal or vaginal microbiome in a corresponding mammal that does not have a RVF. The identifying can include obtaining a sample from the rectum or vagina of the mammal and determining the rectal or vaginal microbiome within the sample. The composition can contain from about 10³ cfu to about 10¹⁵ cfu of each microbe. The composition can be administered orally, vaginally, or rectally. The composition can be formulated as a tablet, a capsule, a pill, a nutritional supplement, or a beverage. The composition can further include one or more prebiotics. The administering can include fecal microbiota transplantation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting rectal and vaginal microbiome profiles in patients with RVF. Brackets under the X-axis denote different subjects, and recurrence is indicated (“Y” or “N”).

FIG. 2A is a Principal Coordinate Analysis graph depicting the relationship of the rectal and vaginal microbiomes. A Weighted UniFrac measure of β-diversity was used, showing significant overlap between the two microbiomes even though they are significantly distinct (p=0.002). Greater between-subject vaginal diversity also was observed (p=0.004). PC1: Principal Coordinate Axis 1; PC2: Principle Coordinate Axis 2. FIG. 2B is a graph plotting differential taxa between the vaginal and rectal microbiomes.

FIGS. 3A and 3B show rectal and vaginal diversity by individual patient. FIG. 3A includes a pair of Principal Coordinate Analysis graphs demonstrating clustering by individual patient more than microbiome site. A Bray-Curtis dissimilarity measure of β-diversity is plotted, and each subject is represented by a different color. PC1: Principal Coordinate Axis 1; PC2: Principle Coordinate Axis 2. FIG. 3B is a pair of graphs showing that the vaginal sample resembles the rectal sample from the same subject at T1 (pre-operative) and T2 (immediate post-operative). The Y-axes denote the Bray-Curtis distance, and a higher number indicates greater dissimilarity. Vag2Vag: between subject distance for the vaginal sample; Rec2Rec: between subject distance for the rectal sample; Rec2Vag: rectal to vaginal distance for the same subject. Rec2Vag was significantly more alike or “clustered” than Vag2Vag or Rec2Rec.

FIG. 4 is a pair of graphs plotting menopausal effects on the rectal (left) and vaginal (right) microbiome. The most significant distance results are given. “Yes” and “No” denote menopausal status. P values denote β-diversity by menopausal status in each plot.

FIG. 5 is a pair of graphs plotting vaginal species richness (observed ASV number) at T1, by depth (left) and by group, rarefied to the minimum depth (right, p=0.07). Bars indicate standard error of the mean. “Yes” indicates recurrence, and “No” indicates non-recurrence. The decrease in vaginal species richness at T1 approximated significance with recurrence.

FIGS. 6A-6C show that the microbiome composition of the vaginal T1 sample is associated with surgical outcome. FIG. 6A is a graph plotting the relative proportion of taxa (indicated by colored bars) that were not (“No”) or were (“Yes”) associated with fistula recurrence. FIG. 6B is a graph plotting Principal Coordinate Analysis based on Bray-Curtis dissimilarity, where “Yes” indicates fistula recurrence and “No” indicates fistula non-recurrence. The p value indicates the distance or difference between the microbiome at T1 of patients who would recur compared to those who did not. FIG. 6C is a graph plotting the relative proportion of each of the 29 taxa differentially present by surgical outcome in the vaginal T1 sample, where “Yes” indicates fistula recurrence and “No” indicates fistula non-recurrence.

FIG. 7 is a pair of graphs plotting the effects of surgery on overall microbiome composition. The colored bars in the left graph depict the relative proportion of taxa at T1 and T2. Taxa are named to the right and are positioned in relative proximity to its color. The graph on the right is a Principal Coordinate Analysis on UniFrac similarity. PC1 and PC2 indicate Principal Coordinates 1 and 2, respectively. “Yes” indicates fistula recurrence, and “No” indicates fistula non-recurrence.

FIG. 8 is a pair of graphs plotting vaginal taxa associated with surgery in patients with non-recurrence (top) and recurrence (bottom) of RVF after surgery. Taxa were identified with a false discovery rate (FDR)<20%.

FIG. 9 is a pair of graphs plotting postoperative (T3) vaginal microbiome diversity relative to T1 in non-recurrent subjects by depth (left) and by group, rarefied to the minimum depth (right, p=0.08). Bars indicate standard error of the mean. “Yes” indicates recurrence, and “No” indicates non-recurrence. The T3 samples trended to reduced diversity relative to the T1 samples in non-recurrent subjects.

FIGS. 10A-10D show that the microbiome composition in the rectal T1 Sample is associated with surgical outcome. FIG. 10A is a series of graphs plotting the Species Richness and Shannon Index (measures of α-diversity) in rectal visit T1 samples, demonstrating that these measures were decreased with recurrence. Species Richness is plotted in the top two graphs. The p-value denotes difference in richness between those who would subsequently recur and those who did not. Shannon Index (a measure of both species richness and evenness) is depicted in the bottom two graphs. The p-value denotes the difference in Shannon Index between those who would recur and those who did not. “Yes” denotes recurrence, and “No” denotes non-recurrence. The left graphs in FIG. 10A are rarefaction curves; these plot the number of species as a function of the sequencing depth. The bars in the right graphs indicated the standard error of the mean. In FIG. 10B, the two columns of colored bars on the left depict the relative proportion of taxa in patients with (“Yes”) and without (“No”) recurrence. The taxa are named on the right and positioned adjacent to their color. FIG. 10C is a graph plotting Principal Coordinate Analysis on Bray-Curtis dissimilarity. PC1: Principal Coordinate 1; PC2: Principal Coordinate 2. “Yes” indicates fistula recurrence, and “No” indicates fistula non-recurrence. FIG. 10D is a graph plotting the relative proportion of each of the 13 taxa differentially present by surgical outcome in the rectal T1 sample. “Yes” indicates fistula recurrence, and “No” indicates fistula non-recurrence.

FIGS. 11A-11C are a set of graphs showing the effect of surgery on the overall rectal microbiome composition (FIG. 11A). Group 1 is the non-recurrent group, while Group 2 subsequently recurred. The P value of 0.008 (FIG. 11B) reflects the overall difference in diversity between T1 and T2. The P value of 0.05 (FIG. 11C) reflects the degree of microbiome difference, or distance, between T1 and T2.

FIG. 12 is a pair of graphs plotting rectal taxa associated with surgery in patients with non-recurrence (top) and recurrence (bottom) of RVF after surgery. Taxa were identified with FDR<20%.

FIG. 13 is a pair of graphs plotting postoperative (T3) rectal diversity relative to T2 in non-recurrent subjects by group (left) and by depth, rarefied to the minimum depth (right, p=0.07). Bars indicate standard error of the mean. The T3 samples trended to increased diversity relative to the T2 samples in non-recurrent subjects.

### DETAILED DESCRIPTION

The microbiome's influence on human health and disease has been investigated through the Human Microbiome Project (Turnbaugh et al., Nature 2007 449(7164):804-810). The study of the microbiome typically relies on unique features of the 16S rDNA gene (found only in Archaea and Bacteria) that can be amplified through polymerase chain reaction (PCR) and used to identify individual organisms. There is evidence for the role of the microbiome in gynecologic and colorectal pathology, but it is not known whether there are pathologic changes in the vaginal and/or rectal microbiome that predispose women for fistula recurrence or prime them for surgical success.

The studies described herein were conducted to characterize the vaginal and rectal microbiome in patients with rectovaginal fistula, including collective and longitudinal evaluation of changes in the microbiome and in quality of life measurements through the perioperative course. As a result, this document provides methods and materials for assessing and/or treating mammals (e.g., humans) having a RVF. For example, methods and materials provided herein can be used to determine if a mammal having a RVF is likely be successfully treated by surgical correction of the RVF, or if the mammal is likely to have a recurrence of the RVF. In some cases, characterization of a rectal and/or vaginal microbiome of a mammal having a RVF can be used to determine if that mammal is likely to experience successful surgical repair, or if the mammal is likely to experience a recurrence. For example, a sample (e.g., a rectal or vaginal sample) obtained from a mammal having a RVF can be assessed to determine if the mammal is likely to be successfully treated with surgery based, at least in part, on the rectal and/or vaginal microbiome of the sample. As described herein, a distinct rectal and/or vaginal microbiome can be present in a mammal that is likely to experience successful surgical repair, and a distinct rectal and/or vaginal microbiome can be present in a mammal that is likely to experience recurrence of the RVF (or another RVF) after surgical repair. This document also provides methods and materials for treating a mammal having a RVF. For example, a treatment for a mammal having a RVF can be selected based, at least in part, on the rectal and/or vaginal microbiome as described herein.

Any type of mammal can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. In some cases, the mammal can be a human. In some cases, a mammal having a RVF can be assessed for whether surgery may be likely to correct the mammal's RVF, or whether the mammal may be likely to experience RVF recurrence after surgical repair (e.g., based, at least in part, on the rectal and/or vaginal microbiome of the mammal).

In some cases, for example, a sample (e.g., a vaginal or rectal sample) obtained from a mammal having a RVF can be assessed to determine whether the mammal may be likely to experience successful surgical correction, or may be likely to experience recurrence after surgery. As described herein, a sample obtained from a mammal having a RVF can be used to determine the vaginal and/or rectal microbiome of the mammal, and can be used to determine whether or not the mammal is likely to experience successful surgical repair.

Any appropriate sample from a mammal (e.g., a human) having a RVF can be assessed as described herein. In some cases, a sample can be a biological sample. For example, a sample can be a rectal or vaginal sample, or a sample taken from the site of a RVF. A sample can be a fresh sample or a fixed (e.g., frozen) sample. Examples of samples that can be assessed as described herein include, without limitation, fecal samples, fluid samples (e.g., vaginal aspirate samples), and tissue samples (e.g., rectal or vaginal tissue biopsies). For example, a fecal sample can be obtained from a mammal having a RVF and can be assessed to determine whether the mammal is likely to experience successful surgical correction or RVF recurrence based, at least in part, on the rectal microbiome of the mammal. For example, a vaginal aspirate sample can be obtained from a mammal having a RVF and can be assessed to determine whether the mammal is likely to experience successful surgical correction or recurrence of the RVF based, at least in part, on the vaginal microbiome of the mammal.

A rectal or vaginal microbiome described herein can include a panel of microbes. A panel of microbes can include any number of microbes. For example, a panel of microbes can include any two or more (e.g., two, three, four, five, six, seven, eight, nine, ten, or more) of the microbes described herein. A microbe in a microbiome described herein can be any type of microbe. In some cases, a microbe can be gram-positive or gram-negative. A microbe can be aerobic or anaerobic. A microbe can belong to any appropriate phylum (e.g., Cyanobacteria, Tenericutes, Actinobacteria, Bacteroidetes, Firmicutes, Fusobacteria, and Proteobacteria). A microbe can belong to any appropriate family or genus (e.g., Bacteroidaceae, Marinifilaceae, Rikenellaceae, Acidaminococcaceae, Christensenellaceae, Ruminococcaceae, Alistipes, Bacteroides, Butyricimonas, Odoribacter, Paraprevotella, Dorea, Faecalibacterium, Lachnospiraceae, Phascolarctobacterium, Subdoligranulum, Parasutterella, and Succinispira). Examples of microbes that can be present in a vaginal and/or rectal microbiome include, without limitation, Sutterella species, Ralstonia species, Escherichia species, Shigella species, Fusobacterium species, Tyzzerella species, Subdoligranulum species, Streptococcus species, Ruminococcus species, Roseburia species, Rumniococcaceae species, Ruminiclostridium species, Phascolarctobacterium species, Peptoniphilus species, Oscillibacter species, Megasphaera species, Lactobacillus species, Lachnoclostridium species, Flavonifractor species, Faecalibacterium species, Ezakiella species, Erysipelatoclostridium species, Dialister species, Clostridium species, Blautia species, Anaerococcus species, Agathobacter species, Prevotella species, Porphyromonoas species, Parabacteroides species, Barnesiella species, Bacteroides species, Alistipes species, Mobiluncus species, Collinsella species, and Atopobium species. In some cases, microbes that can be present in a vaginal microbiome described herein can be as shown in TABLE 5. In some cases, microbes that can be present in a rectal microbiome described herein can be as shown in TABLE 6.

Any appropriate method can be used to identify the presence or absence of one or more microbes described herein (e.g., one or more microbes in a rectal or vaginal microbiome described herein). For example, 16S rRNA-based techniques, next-generation sequencing, shotgun metagenomics, and fluorescence in situ hybridization can be used to identify the presence or absence of one or more microbes. In some cases, one or more microbes can be identified as described in Example 1.

In some cases, the characteristics of a microbiome can be assessed to determine whether a mammal having a RVF is likely to experience a successful surgical correction, or whether the mammal is likely to experience recurrence after surgery. For example, the diversity (also referred to as “richness”) and abundance of the species present in a microbiome can be determined. In some cases, a mammal having an RVF can be identified as having a vaginal and/or rectal microbiome with a reduced level of diversity (the number of different strains present in a microbiome), or a reduced abundance of certain taxa of microbes (e.g., Firmicutes or Bacteroidetes). A reduced level of the diversity of a sample or the abundance of a microbe refers to a level that is lower than the median level typically observed in a sample (e.g., a control sample) from one or more healthy mammals (e.g., healthy humans and mammals that do not have a RVF). An eliminated level of a microbe refers to any non-detectable level of that microbe. A reduced level of diversity or abundance can be any level that is less (e.g., at least 5% less, at least 10% less, at least 20% less, at least 25% less, at least 50% less, or at least 75% less) than a control level. The diversity of a microbiome and the abundance of one or more microbes within a microbiome can be assessed using any suitable method (e.g., a 16S rRNA-based technique, next-generation sequencing, shotgun metagenomics, and/or fluorescence in situ hybridization).

In some cases, a mammal can be identified as being more likely to experience recurrence of RVF after surgical correction based, at least in part, on the mammal's identification as having a rectal or vaginal microbiome with reduced diversity or reduced abundance of certain taxa. In some cases, the likelihood of successful surgical correction (without recurrence) of a RVF in such a mammal can be increased by modulating the rectal and/or vaginal microbiome. For example, a microbiome can be modulated to increase its diversity and/or the abundance of particular taxa in the microbiome.

Having the ability to identify mammals as being more likely to experience recurrence of a RVF after surgical correction can allow those mammals to be and treated in an effective and reliable manner with probiotic compositions prior to surgery in order to increase the likelihood of successful surgical RVF correction, without recurrence. For example, the microbiome-modulating compositions described herein can be used to treat patients identified as having RVF and having a microbiome with reduced diversity or abundance of particular taxa with a composition containing one or more microbial strains as described herein.

This document also provides methods for treating a mammal having an RVF, to modulate the microbiome of the mammal. In some cases, the methods also can include determining the rectal and/or vaginal microbiome of the mammal. When treating a mammal (e.g., a human) having a RVF as described herein, the treatment can be effective to modulate the rectal and/or vaginal microbiome. For example, the treatment can be effective to modulate the rectal and/or vaginal microbiome by increasing the diversity of species therein or by increasing the abundance of organisms within particular taxa (e.g., phyla such as Cyanobacteria, Tenericutes, Bacteroidetes, Firmicutes, and Proteobacteria, or genera or families such as Bacteroidaceae, Marinifilaceae, Rikenellaceae, Acidaminococcaceae, Christensenellaceae, Ruminococcaceae, Alistipes, Bacteroides, Butyricimonas, Odoribacter, Paraprevotella, Dorea, Faecalibacterium, Lachnospiraceae, Phascolarctobacterium, Subdoligranulum, Parasutterella, and Succinispira).

“Increased diversity” with respect to a microbiome can be any increase in the number of different species detected (e.g., using a 16S rRNA-based technique, next-generation sequencing, shotgun metagenomics, and/or fluorescence in situ hybridization) in the microbiome, as compared to the number of different species detected prior to modulation. In some cases, for example, “increased diversity” refers to an increase of at least 5% (e.g., at least 10%, at least 20%, at least 30%, at least 50%, or at least 100%) in the number of different species present at detectable levels in a microbiome, as compared to the number of different species that were present at detectable levels in the microbiome prior to modulation. In some cases, an increase in the diversity of a microbiome can result in the presence of a detectable level of at least one (e.g., two, three, four, five, or more than five) species from at least ten (e.g., at least 12, at least 15, or all 18) of the following genera and families: Bacteroidaceae, Marinifilaceae, Rikenellaceae, Acidaminococcaceae, Christensenellaceae, Ruminococcaceae, Alistipes, Bacteroides, Butyricimonas, Odoribacter, Paraprevotella, Dorea, Faecalibacterium, Lachnospiraceae, Phascolarctobacterium, Subdoligranulum, Parasutterella, and Succinispira.

“Increased abundance” with respect to a particular organism or group of organisms refers to an increase of at least 5% (e.g., at least 10%, at least 20%, at least 30%, at least 50%, or at least 100%) in the detected amount of that organism or group of organisms. In some cases, an increase in the abundance of a particular microbe can be a statistically significant increase (e.g., p<0.05) in the detected level of the microbe, as compared to the detected level of the microbe of prior to modulation.

Modification of the vaginal and/or rectal microbiome can be achieved through a variety of treatment approaches, including oral or vaginal administration of probiotics, prebiotics, or synbiotics, or fecal microbiota transplantation. In some cases, the methods provided herein can include administering to a mammal, or instructing a mammal to self-administer, a composition containing one or more microbial strains. For example, a composition can include one or more (e.g., two, three, four, five, six, seven, eight, nine, 10, or more than 10) microbial strains selected from Cyanobacteria, Tenericutes, Bacteroidetes, Firmicutes, Proteobacteria, and combinations thereof. In some cases, a composition can include one or more (e.g., two, three, four, five, six, seven, eight, nine, 10, or more than 10) microbial strains selected from Bacteroidaceae species, Marinifilaceae species, Rikenellaceae species, Acidaminococcaceae species, Christensenellaceae species, Ruminococcaceae species, Alistipes species, Bacteroides species, Butyricimonas species, Odoribacter species, Paraprevotella species, Dorea species, Faecalibacterium species, Lachnospiraceae species, Phascolarctobacterium species, Subdoligranulum species, Parasutterella species, and Succinispira species.

A composition containing one or more microbial strains can include any appropriate amount of each strain. In some cases, for example, a composition containing one or more microbes can be formulated in a dose such that a mammal receives from about 10³ to about 10¹⁵ cfu (e.g., about 10³ to about 10⁵, about 10⁵ to about 10⁷, about 10⁷ to about 10⁹, about 10⁹ to about 10¹¹ cfu, about 10¹¹ to about 10¹³ cfu, or about 10¹³ to about 10¹⁵ cfu) of each microbe present in the composition. A composition used in the methods described herein can be in the form of an oral medicament or nutritional supplement, or in the form of a medicament for rectal or vaginal administration. For example, compositions for oral administration can be in the form of a pill, tablet, powder, liquid, or capsule. Tablets or capsules can be prepared with pharmaceutically acceptable excipients such as binding agents, fillers, lubricants, disintegrants, or wetting agents. In some cases, tablets can be coated. In some cases, a composition containing at least one microbial strain can be formulated such that the microbes are encapsulated for release within the intestines of a mammal. Liquid preparations for oral administration can take the form of, for example, solutions, syrups, or suspension, or they can be presented as a dry product for constitution with saline or other suitable liquid vehicle before use. For example, a composition provided herein can be in the form of a food product formulated to contain at least one microbial strain having a desired activity. Examples of such food products include, without limitation, milk (e.g., acidified milk), yogurt, milk powder, tea, juice, beverages, candies, chocolates, chewable bars, cookies, wafers, crackers, cereals, treats, and combinations thereof. A composition for rectal administration can be in the form of a suppository, or an enema, for example.

In some cases, a composition containing or more one microbial strains also can include a pharmaceutically acceptable carrier for administration to a mammal. Suitable pharmaceutically acceptable carriers include, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other known carriers for oral or rectal administration.

In some cases, a composition containing one or more microbial strains also can include one or more (e.g., two, three, four, five, or more than five) prebiotics. Prebiotic compounds that can be included in a composition for use in the methods described herein can include, without limitation, one or more oligosaccharides (e.g., plant-derived fructans and/or galactans), anti-oxidants, fructooligosaccarides (FOS), short-chain fatty acids (e.g., butyrate, acetate, and/or propionate), dietary fibers (e.g., inulin), flavonoids, resistant starch, beta-glucan, pectin, glucomannan, cellulose, lignin, arabinoxylan oligosaccharides (AXOS), or any combination thereof.

In some cases, a composition containing one or more microbial strains described above also can include one or more additional, probiotic strains (e.g., two, three, four, five, or more than five) probiotics. Additional probiotic strains that can be included in a composition for use in the methods described herein can include, without limitation, one or more of the differentially abundant taxa identified as being present in non-recurrent patients in the studies described herein. For example, a composition can include one or more additional probiotic strains selected from Ruminococcaceae species, Bacteroidaceae species, Acidaminococcaceae species, Rikenellaceae species, Marinifilaceae species, Christensenellaceae species, Clostridiales species, Bacteroides species, Faecalibacterium species, Phascolaractobacterium species, Alistipes species, Subdoligranumum species, Ruminococcus species, Parasutterella species, Paraprevotella species, Lachnospiraceae species, Odoribacter species, Dorea species, Butyricimonas species, Succinispira species, and combinations thereof.

In addition, microbiome modulation can be achieved in a mammal having a RVF by fecal microbiota transplantation. In general, this method includes making stool from a healthy donor into a liquid mixture, and then transferring the mixture into the colon of a recipient. For example, a donor stool sample can be transferred into the colon of a mammal having a RVF in order to reintroduce or boost the number or variety of microbes in the mammal.

The invention will be further described in the following examples, which do not limit the scope of the invention described in the claims.

### EXAMPLES

### Example 1—Materials and Methods

The presently described study involved the longitudinal collection of vaginal and rectal microbial samples from women with RVF who were planning to undergo surgical treatment during an 18 month window. Only patients with diagnosed RVF were enrolled; women with colovaginal or enterovaginal fistula were not included in the study. Additional exclusion criteria included: current pregnancy, current or recent history (within past 4 weeks) of antibiotic use, history of chemotherapy within two years of fistula surgery, prior pelvic or abdominal radiation therapy, and current or planned intestinal diversion. Patient demographic information was collected and included age, BMI, menopausal status, and tobacco use. Fistula size, etiology, location and history of prior repair also were documented. Fistula location was designated as low, mid or high (Das et al., Clin Colon Rectal Surg 2016, 29:50-56) at the time of clinical examination. The PROMIS-10 (Patient-Reported Outcomes Measurement Information System) Global Health tool was completed by each patient at enrollment and once again at a 6-8 week postoperative visit. The PROMIS-10 tool is a validated, 10 item measure of mental and physical health-related quality of life that has been used to assess outcomes in several surgical disciplines (Jones and Stukenborg, J Am Coll Surg 2017, 224(3):245-254).

Microbial samples were obtained from rectal and vaginal locations at several time points for each patient. Pre-operative (T1) vaginal and rectal samples were collected in clinic within four weeks of surgery or in the operating suite prior to initiation of intravenous antibiotics and surgical site preparation. Immediate post-operative (T2) samples were collected by the surgeon at the time of surgical site closure, prior to the patient leaving the operating room. This collection occurred after administration of prophylactic antibiotics, betadine surgical site prep, and intraoperative use of irrigation; mechanical bowel prep was not utilized. Finally, samples were collected at the postoperative visit 6-8 weeks following surgery (T3), or earlier if fistula recurrence occurred (T3R). A patient interview was completed 12 weeks after surgery to conclude study participation. One subject did not return for a postoperative visit, so no T3 samples were collected from that subject.

For each collection, two sterile, polyester tipped swabs were held together and rotated over the site of the fistula within each environment (i.e., vagina or rectum). Swabs were placed in HOLOGIC® Aptima transport tubes containing 2.9 mL of media and flash frozen to −70° C. and stored within 15 minutes of collection. Samples were accessioned into a Research and Laboratory Information Management System (RLIMS) at this time for tracking purposes. Once all specimens were collected, the de-identified samples were sent to the University of Minnesota Genomics Center for DNA extraction and 16S rDNA processing/sequencing. The amount of DNA extracted from each sample was quantified using the Qubit dsDNA HS (High Sensitivity) Assay Kit. The DNA samples were then used to partially (V3-V5) amplify the microbial 16S rDNA genes through PCR. Controls of both the DNA extraction and microbiome enrichment processes were performed. One vaginal and one rectal sample had a sequencing depth similar to the negative controls and were excluded from analysis. The PCR product was subsequently purified using AGENCOURT® AMPURE® and quantified before sequencing. 16S rDNA hypervariable tag sequencing was performed using a high throughput next-generation Illumina MiSeq (San Diego, Calif.) sequencing platform.

Bioinformatics processing of the 16S rDNA amplicon sequence reads was accomplished with DADA2 (Callahan et al., Nature Methods 2016, 13(7):581), which is a software package that models and corrects amplicon errors. This effectively “de-noised” the sequence reads to identify ASV (amplicon sequence variants) of true biological origin. The SILVA ribosomal RNA gene database project (Quast et al., Nucleic Acids Res 2012, 41(D1):D590-D596) was used to assign taxonomic lineage based on Naïve Bayes Classifier. Finally, FastTree (Price et al., PloS ONE 2010, 5(3):9490), a bioinformatics method for constructing large phylogenies and for estimating their reliability, was used to construct the phylogenetic tree among ASVs. A total of 5,531,556 sequence reads (2,586-137,573 reads per sample) were obtained (mean of 67,458 reads) after quality control.

To analyze the data (ASV table), measures of α-diversity, β-diversity, and taxa abundance were used. α-diversity is used to measure the diversity within a sample. In the present studies, two different metrics were used to calculate α-diversity. The first metric was the count of the number of different microbes (ASV count) in a sample (referred to as “species richness”). The second metric, the Shannon Index, measures the richness in a sample as well as the distribution of different microbes within that sample (referred to as “evenness”). Evenness is a measure of the variation in the relative abundance of different species in a sample. Since the a-diversity of a sample depends on the sequence depth (the total number of reads), sample reads were rarefied to an equal depth in order to be comparable. These indices are presented graphically with rarefaction plots, which plot the number of ASVs versus the number of reads sampled. Linear models or linear mixed effect models were used for testing the association of α-diversity with variables of interest (e.g., recurrence status) while adjusting for potential confounders (e.g., BMI).

The second measure, β-diversity, is a term for the comparison of samples to each other. β-diversity provides a measure of the ecological distance or dissimilarity between bacterial communities. Four different β-diversity measures (weighted UniFrac, unweighted UniFrac, generalized UniFrac (α=0.5), and Bray-Curtis) were calculated, each providing a unique view of community structure (Chen et al., Bioinformatics 2012, 28(16):2106-2113). Weighted UniFrac measures the abundance of observed ASVs and phylogeny, while unweighted UniFrac only compares the presence or absence of ASVs and phylogeny. The generalized UniFrac unifies the weighted and unweighted UniFrac distance into a single framework, while the Bray-Curtis distance is a non-phylogeny based method that also takes abundance into account. Rarefaction was performed on the ASV table before calculating these distances. The PERMANOVA test based on β-diversity measures was used to test for associations between the overall microbiota composition and variables of interest, and PERDISP was used for testing the difference in dispersion (i.e., between-subject variability) between groups (both 999 permutations) (Chen et al., supra). Within-subject permutation was used for the comparison within the subjects. Principal Coordinate Analysis plots (PCoA) were used to graphically demonstrate this data. These plots project the distance matrix into a new set of orthogonal axes on which the data are presented. Typically, the first two axes (called PC1 are PC2) can be used to depict or explain the maximum amount of variation in the data.

Finally, taxon-level differential abundance analysis was performed at the phylum, class, order, family, and genus level to identify differences in the specific microbial genera of each collected cohort. Taxa with prevalence less than 10% or with a maximum proportion less than 0.2% were excluded to reduce the number of tests. The count data was normalized into relative abundances by the GMPR (Chen et al., PeerJ 2018, 6:e4600) approach to address variable sequencing depth. To identify differentially abundant taxa associated with variables of interest while adjusting for potential confounders, a multiple linear regression was fit based on the square-root transformed normalized abundance. Permutation (999 permutations) based on the F-statistic was used to assess the significance in order to address potential non-normality of the data. Within subject permutation was used for the comparison within the subjects. False discovery rate (FDR) control (B-H procedure), which controls the percentage of the false positives in the claimed positives, was used to correct for multiple testing at each taxonomic level, and FDR-adjusted p- or q-values <0.20 were considered significant (i.e., the expected percentage of the false positives in the result is less than 20%). The relatively large FDR cutoff was used so as not to miss differential low-abundance bacteria, which usually have low statistical power.

### Example 2—Results

A total of 14 patients were enrolled into the study over a period of 18 months. Demographic and baseline fistula information are presented in TABLE 1. There were 8 fistula recurrences following surgery; all fistulas were of the “low” classification (Das et al., supra). All surgical repairs included excision of the fistula tract with closure. Concomitant surgeries included the use of a flap in 4 cases (3 Martius, 1 Gracilis), an endorectal advancement flap in 1 case, and overlapping sphincteroplasty in 4 cases. Complications included one Martius flap breakdown without fistula recurrence and one sphincteroplasty breakdown that required return to the OR for repair and bowel diversion. Six of the 8 recurrences had an average of 3 prior repair attempts before the present study. The only demographic variable that was statistically significant between those who experienced recurrence and those who did not was BMI (mean 33.1 vs 25.6 respectively, p=0.02). PROMIS quality of life scores are shown in TABLE 2. There were significant differences in post-op physical and mental health scores between patients with and without fistula recurrence (p=0.006 and p=0.023 respectively). No differences were appreciated preoperatively between groups.

### Microbiome Characterization

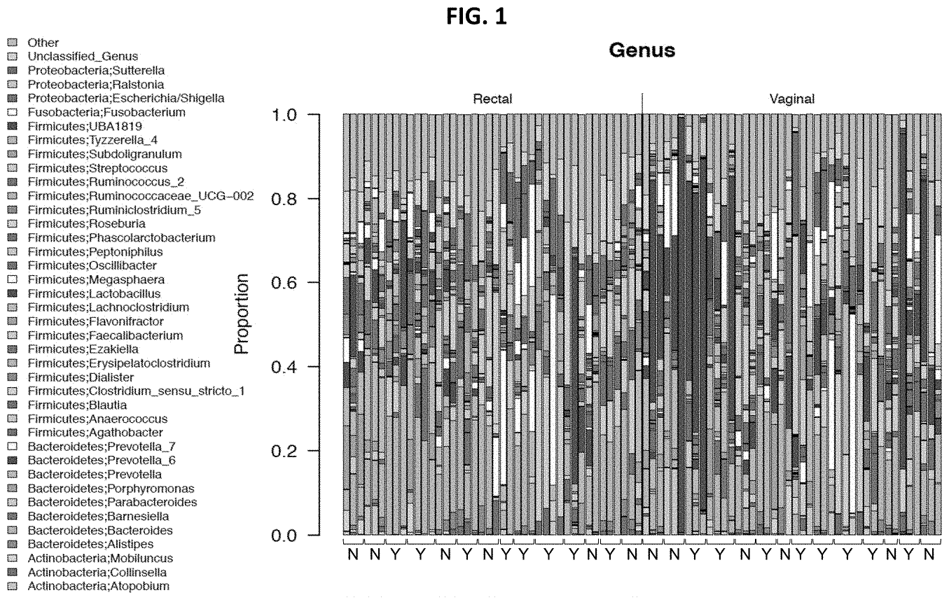

The distribution of the samples after quality control is shown in TABLE 3. Deep sequencing of the V3-V5 16S rDNA region of all 82 samples resulted in the analysis of 16,723 ASVs belonging to 15 phyla, 85 families, and 339 genera. The dominant taxa of the vaginal microbiome included Lactobacillus (16.2%), Bacteroides (9.4%) and Prevotella (6.4%), and the dominant rectal taxa included Bacteroides (14.6%), Parabacteroides (5.8%), Prevotella (5.5%), and Faecalibacterium (5.4%) (FIG. 1).

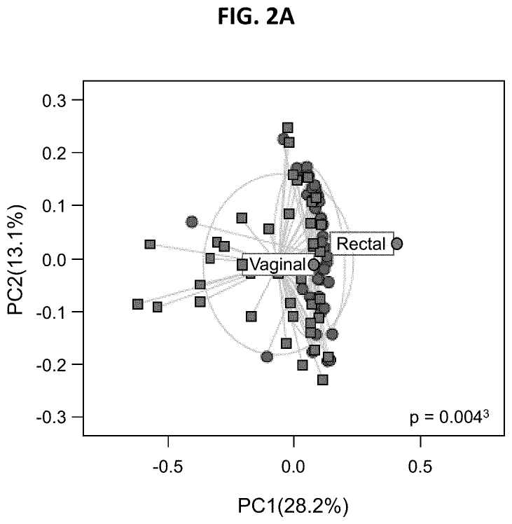

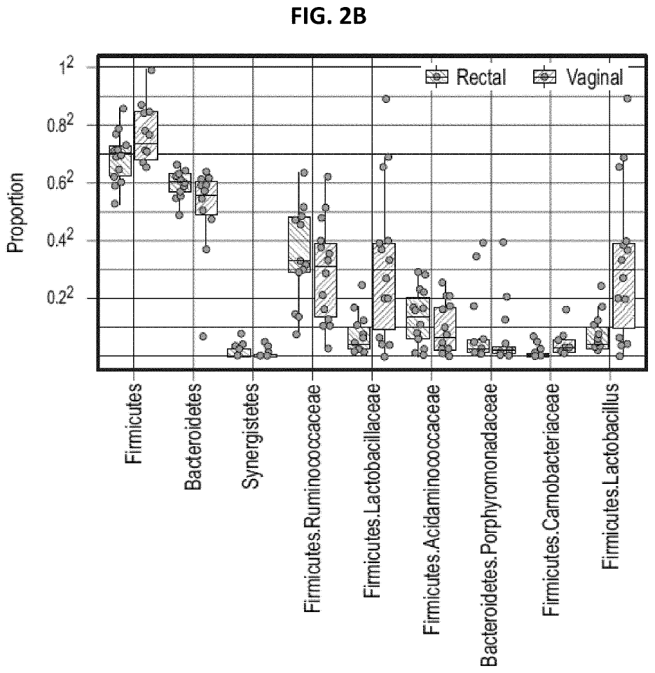

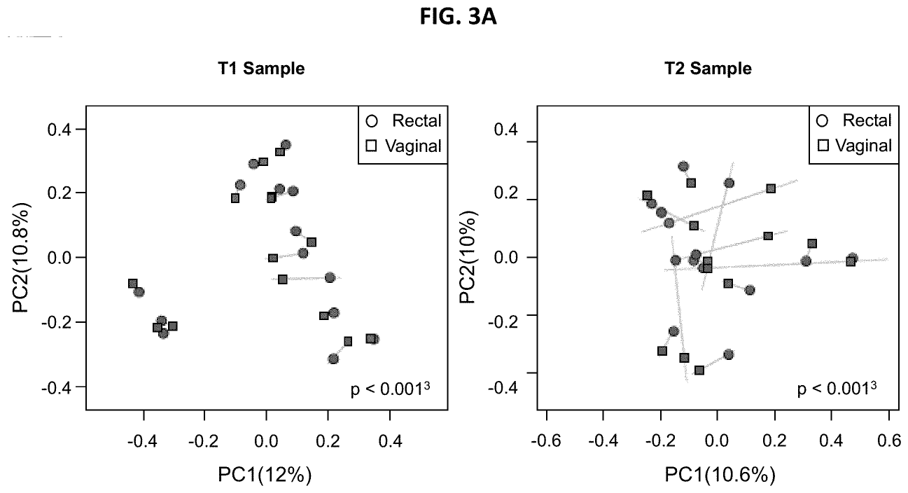

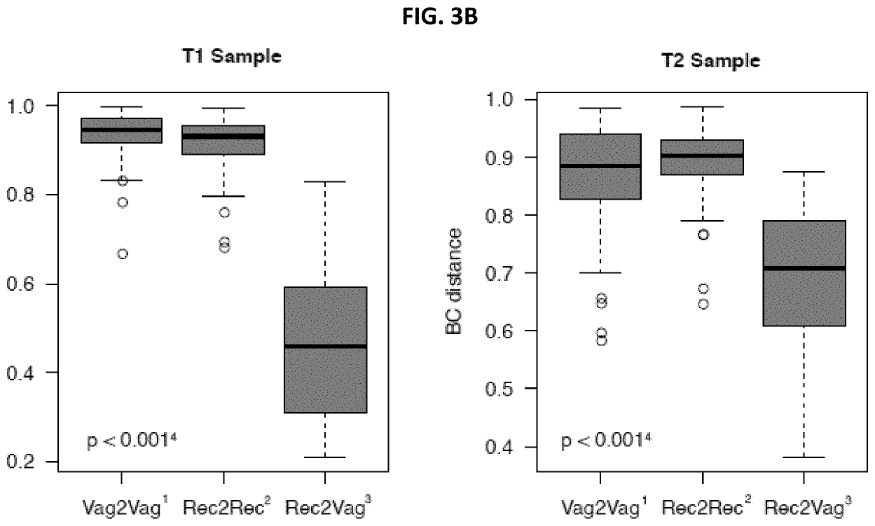

Several unusual relationships were identified that had not previously been described. First, a PCoA plot based on the weighted UniFrac distance (FIG. 2A) showed a significant degree of overlap and similarity between the vaginal and rectal microbiome across the cohort, with the vaginal microbiome demonstrating greater between-sample diversity (p=0.004, PERDISP). Some taxa, including Lactobacillus, remained differential between the vaginal and rectal microbiome (FIG. 2B). The Bray-Curtis distances looking at samples collectively at T1 and T2 showed microbiome clustering significantly more by patient than by site (FIG. 3A), meaning that the vaginal microbiome of an individual is more similar to their own rectal microbiome than to the vaginal microbiome of the remaining cohort (and vice versa) (FIG. 3B). Typically, in patients without fistula, the rectum has much greater microbial diversity than the vagina, there is little overlap between the two, and clustering occurs more by site than by the individual.

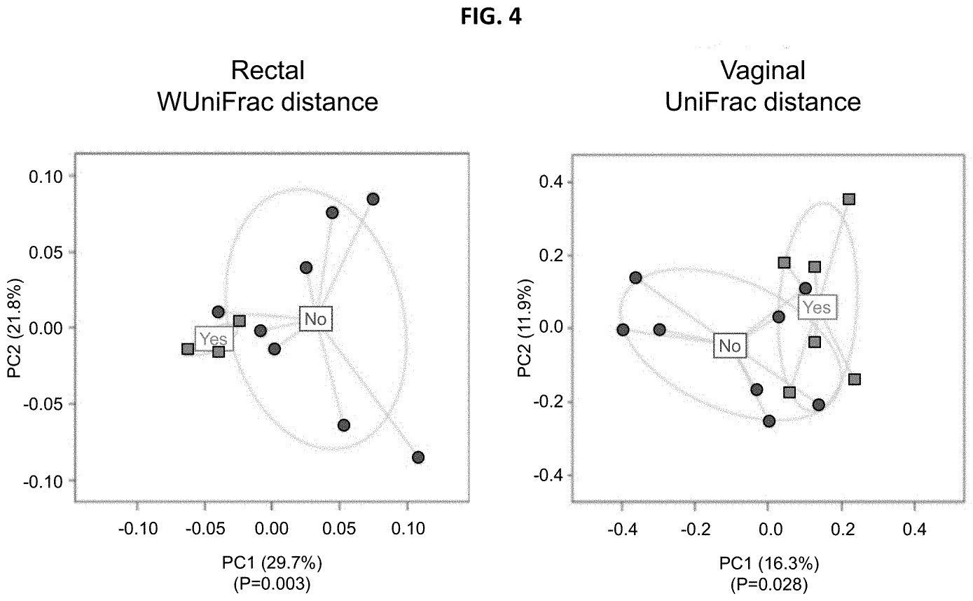

PERMANOVA analysis revealed evidence of microbiome association for several clinical variables for both the vaginal and rectal samples. These included menopausal status, BMI, and history of vaginal infection in the past six months (TABLE 4 and FIG. 4). Of these associations, BMI again was the only measure that was significantly different between patients who recurred and those who did not (p=0.029). This potential confounder was therefore adjusted for when the rectal and vaginal microbiota were compared between the two groups.

### Vaginal Microbiome

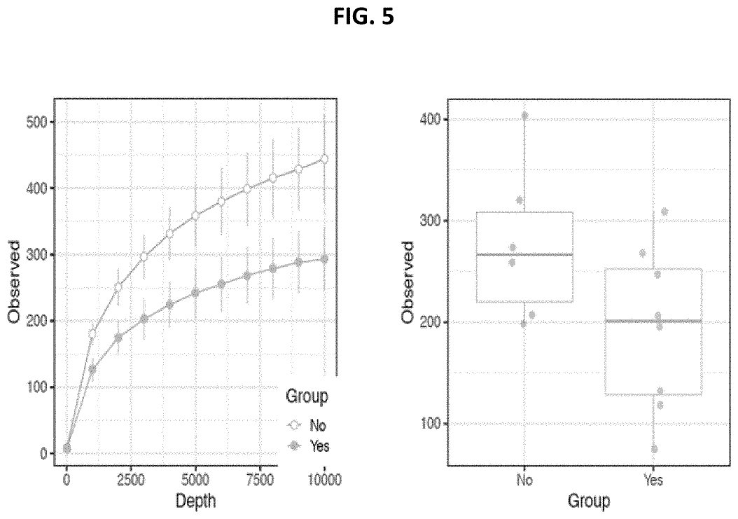

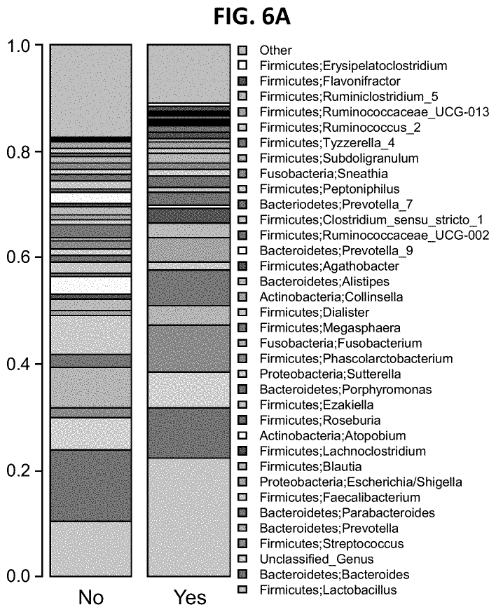

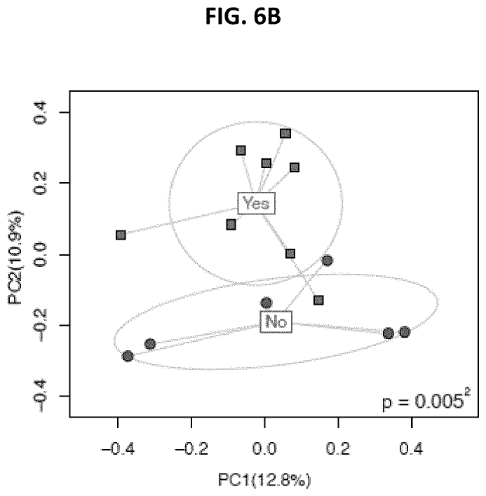

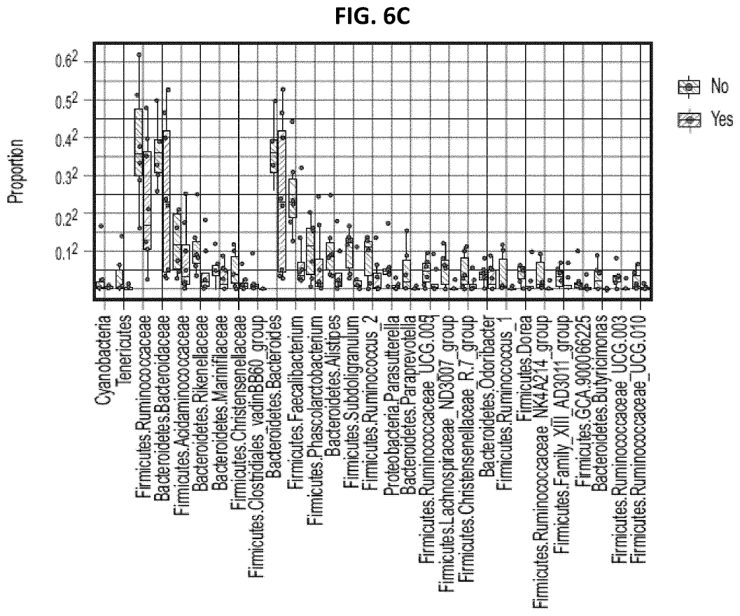

A comparative analysis of vaginal samples was performed at each collection time point for those with and without fistula recurrence. Reduced species richness (the number of observed ASVs) approximating significance was seen preoperatively (T1) in patients who would subsequently recur as compared to those who would not (p=0.07; FIG. 5). FIG. 6A demonstrates the relative proportion of taxa after adjusting for BMI. The overall microbiota composition was found to be markedly different between the two groups, as revealed by the β-diversity analysis (Bray-Curtis distance, p=0.005, omnibus p-value=0.013; FIG. 6B). Differential abundance analysis revealed 29 different taxa that were depleted in patients with recurrence (FIG. 6C). The most significant included the Firmicutes Subdoligranulum, Ruminococcaceaea UCG-010, and Ruminococcaceaea NK4A214 group, as well as the Bacteroidetes Alistipes, and Rikenellaceae (TABLE 5).

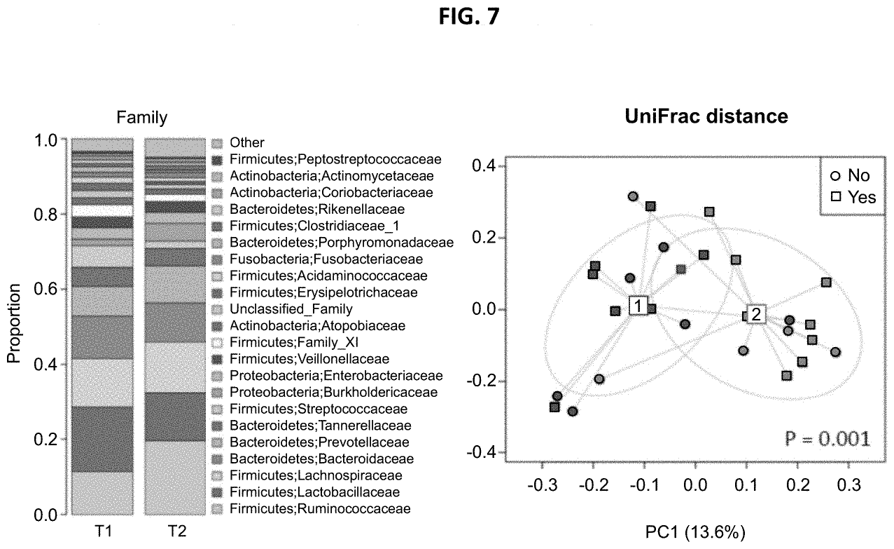

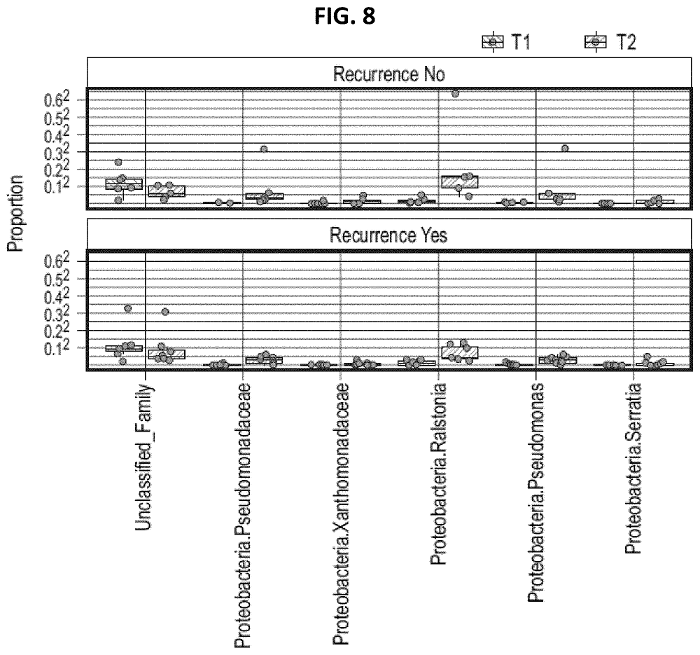

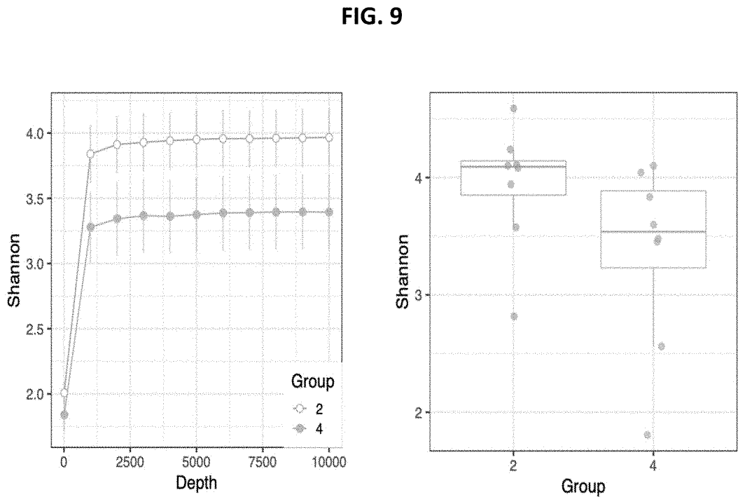

When comparing the T1 vs. T2 collections based on the β-diversity, it was observed that surgery changed the overall vaginal microbiome composition (UniFrac, p=0.001; FIGS. 7 and 8). The greatest effect was seen with Pseudomonadaceae, Xanthomonadaceae, Pseudomonas, Ralstonia, and Serratia, all of which were decreased in patients with subsequent fistula recurrence. Because these species were present at similar levels in both recurrent and non-recurrent T1 samples, it appears that the surgical effect on T2 may not be related to recurrence. There were no significant differences in recurrent vs non-recurrent samples collected at T2. Further analysis of non-recurrent subjects demonstrated a trend toward increased species diversity of T1 relative to T3 (p=0.07; FIG. 9); no difference in diversity between T2 and T3 was detected at this power.

### Rectal Microbiome

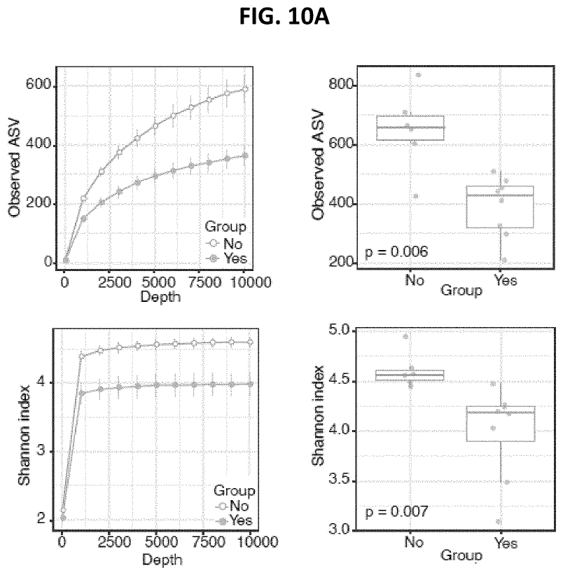

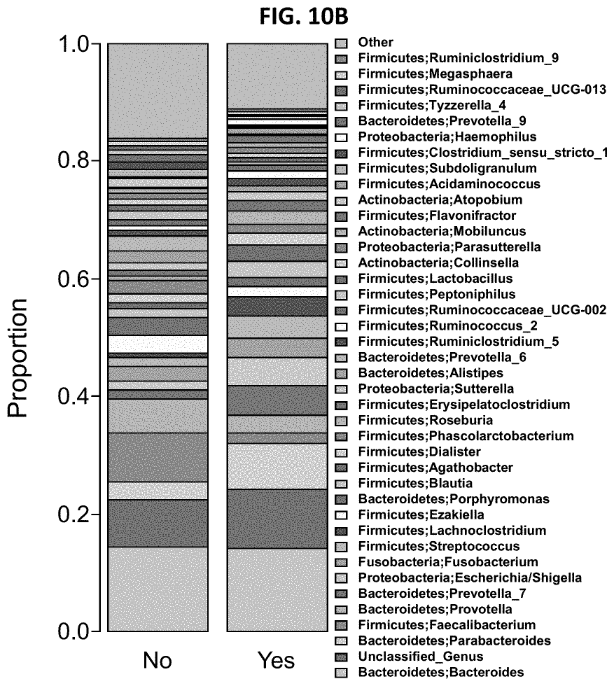

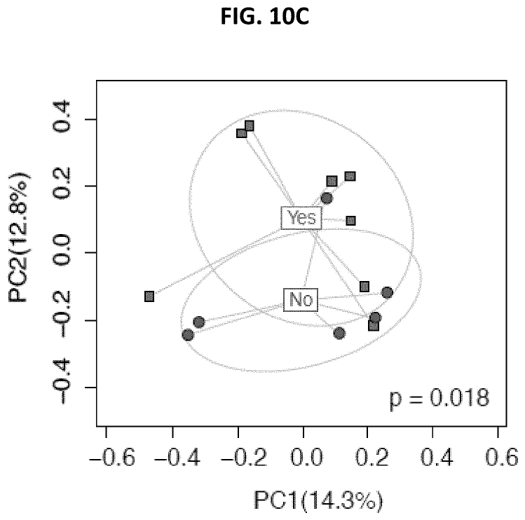

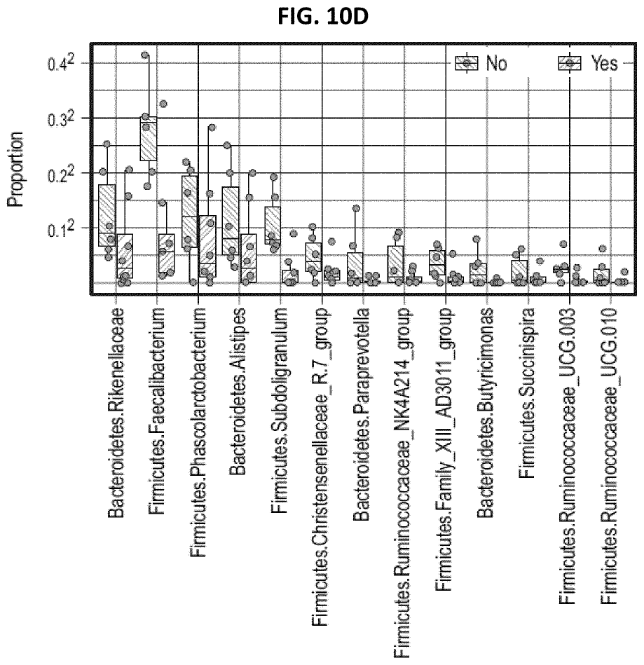

A comparative analysis of the recurrent vs. non-recurrent rectal samples at each collection time point after BMI adjustment also demonstrated a significant reduction in the α-diversity measures of species richness/Shannon index from T1 samples of patients who would subsequently recur, compared to those who would not (FIG. 10A). The relative proportion of taxa are depicted in FIG. 10B. Like the vaginal samples, the overall microbial composition was different between the two groups as revealed by the β-diversity analysis (Bray-Curtis distance, p=0.018; FIG. 10C). Differential abundance analysis revealed 13 different taxa that were depleted in patients with recurrence; these showed significant overlap with those depleted in the vaginal samples, with 11 of the 13 taxa in common (FIG. 10D and TABLE 6).

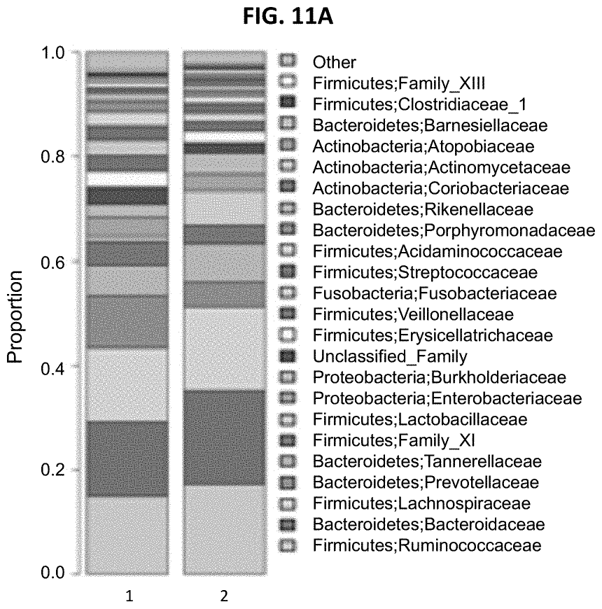

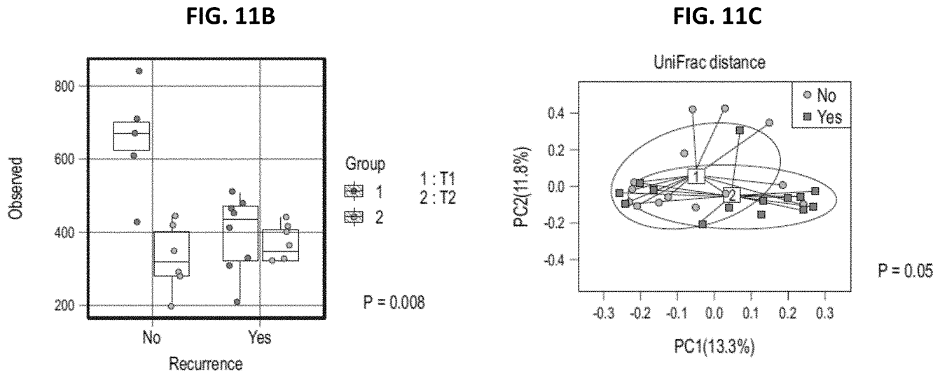

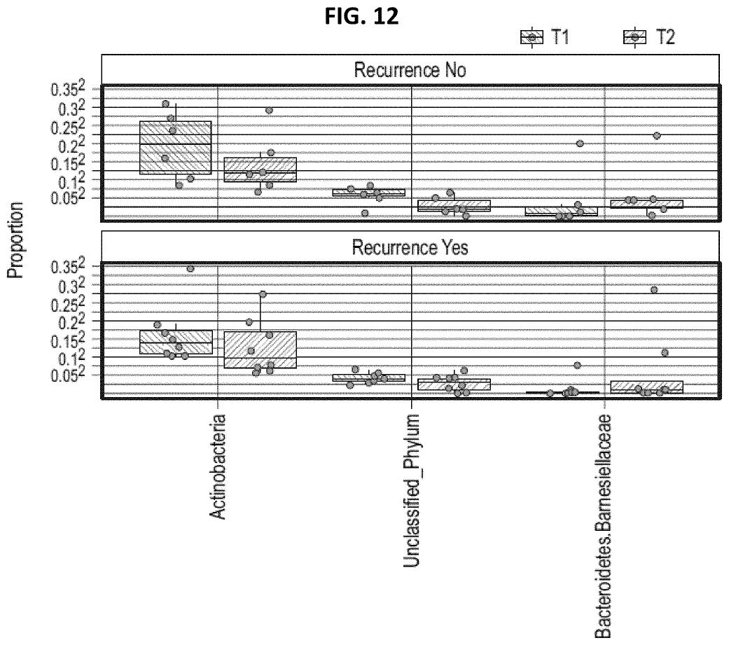

It was again noted that surgery was associated with reduced species richness at T2 compared to T1 (FIG. 11A), especially for non-recurrent samples (p=0.008; FIG. 11B). The P value of 0.05 reflected the degree of microbiome difference (or distance) between T1 and T2 (FIG. 11C). The overall microbiome change affected Actinobacteria and Barnesiellaceae most significantly (FIG. 12). No difference in T2 samples between the two groups was observed.

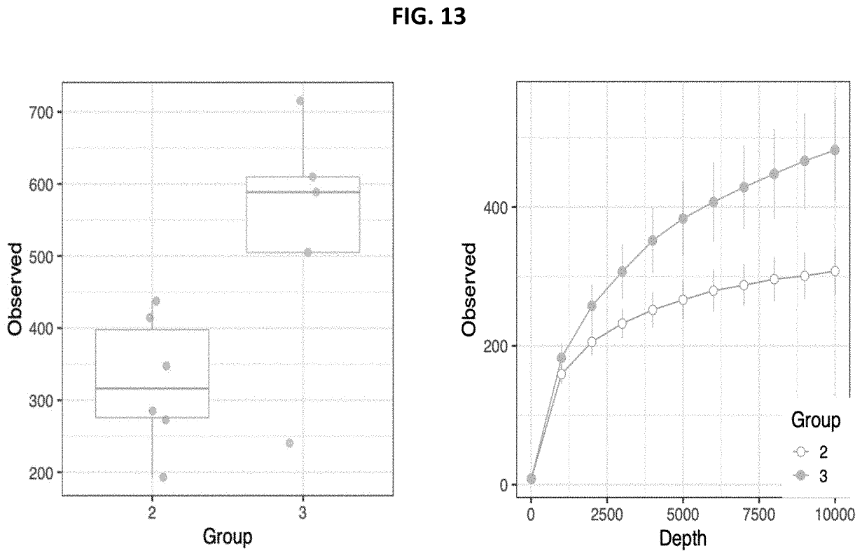

In the non-recurrent rectal cohort, increased diversity in T3 relative to T2 (species richness, p=0.07; FIG. 13) approximated significance, but increased diversity in T3 relative to T1 did not. This is the opposite of what was seen in the vaginal cohort. Rectal diversity was similarly decreased in all recurrent samples.

Thus, the studies described herein present a prospective cohort study in which the rectal and vaginal microbiome was characterized for the first time in patients with RVF. The results of these studies indicated that increased diversity at both sites is protective from recurrence, that there is greater between-subject microbial variation vaginally than rectally, and that the microbiome clusters more by individual than by site. The observation of greater microbial variation across the cohort was unexpected, as the opposite is typically true (Human Microbiome Project Consortium, Nature 2012, 486:207-214). Even more surprising was the increase in vaginal diversity associated with a favorable clinical outcome, as this (unlike rectal diversity) is not typical.

The dominant taxa of the vaginal microbiome (recurrent or otherwise) was not characteristic of the general population (Hyman et al., Proc Natl Acad Sci USA 2005, 102(22):7952-7957). The vagina typically has relatively low microbial diversity due to a predominance of lactobacillus, which may confer protection and overall health in the vaginal environment (Kim et al., J Clin Microbiol 2009, 47(4):1181-1189; Ma et al., Annu Rev Microbiol 2012, 66:371-389; Hyman et al., supra; Ravel et al., Proc Natl Acad Sci USA 2011, 108(S1):4680-4687; and Muhleisen and Herbst-Kralovetz, Maturitas 2016, 91:42-50). Pathologic processes are conversely characterized by increased diversity in the setting of reduced or absent lactobacillus and a concomitant exponential increase in the concentrations of other bacteria such as Prevotella, Gardnerella, Mycoplasma, and Atopobium (Verhelst et al., BMC Microbiology 2004, 4:16; and Ferris et al., BMC Infec Dis 2004, 4:5). None of these bacteria contributed to the 29 differentially abundant taxa associated with fistula recurrence in the present study population.

Increased rectal microbial diversity, unlike in the vaginal microbiome, typically is a marker of health and better outcomes (Human Microbiome Project Consortium, Nature 2012, 486:207-214; Chang et al., J Infect Dis 2008, 197(3):435-438; Ott et al., Gut 2004, 53(5):685-693; and Gong et al., Gastroenterol Res Pract 2016, 6951091). Several taxa were associated with surgical success, including significantly higher levels of Firmicutes Faecalibacterium (which has anti-inflammatory properties; (Verhoog et al., Nutrients 2019, 11(7):1565), Ruminococcus, and Christensenellaceae, as well as Bacteroides Alistipes and Rikenellaceae. These organisms have been associated with improved outcomes in diverse conditions ranging from Inflammatory Bowel Disease, HIV, Non-Hodgkins Lymphoma, and melanoma (Gong et al., supra; Verhoog et al., supra; Montassier et al., Genome Medicine 2016, 8:49; Dinh et al., J Infect Dis 2015, 211(1):19-27; Mutlu et al., PLoS Pathog 2014, 10(2):e1003829; and Dubin et al., Nat Commun 2016, 7:10391). All of these were differentially abundant in the study specimens (TABLES 5 and 6).

These studies also showed that T1 and T3 samples had similarly increased diversity, while T2 samples remained distinct from both. This suggests a longitudinal return in diversity to mimic the T1 milieu by the time T3 was collected 6 to 8 weeks after surgery. Decreased diversity persisted through all three samples collected in patients that would recur (T1, T2, and T3R).

These studies indicated that there was a strong microbial influence on the successful repair of RVF. Organisms with known antimicrobial and anti-inflammatory properties were present in higher proportions in patients who did not experience recurrence; these organisms may counter the pro-inflammatory RVF milieu and improve the post-surgical healing process. The composition of the rectal microbiome with its significantly higher microbial burden and higher-pressure environment likely facilitates the establishment and persistence of an atypically diverse vaginal microbiome. The diversity of both areas may be a key modulator of surgical success.

TABLE 1
Patient Demographics and Baseline Fistula Characteristics

| | No Recurrence | | Recurrence | | Total | | |
|---|---|---|---|---|---|---|---|
| | (N = 6) | | (N = 8) | | (N = 14) | | p value |
| Age | | | | | | | 0.7657 |
| Mean (SD) | 43.5 | (19.1) | 50.4 | (10.4) | 47.4 | (14.5) | |
| Range | (23.0-71.0) | | (38.0-68.0) | | (23.0-71.0) | | |
| BMI | | | | | | | 0.0210 |
| Mean (SD) | 25.6 | (5.1) | 33.1 | (5.2) | 29.9 | (6.3) | |
| Range | (19.5-34.7) | | (24.6-42.8) | | (19.5-42.8) | | |
| Smoker | | | | | | | 1.0000 |
| Current | 1 | (16.7%) | 2 | (25.0%) | 3 | (21.4%) | |
| Never | 2 | (33.3%) | 3 | (37.5%) | 5 | (35.7%) | |
| Prior | 3 | (50.0%) | 3 | (37.5%) | 6 | (42.9%) | |
| Menopausal | | | | | | | 0.6270 |
| No | 4 | (66.7%) | 4 | (50.0%) | 8 | (57.1%) | |
| Yes | 2 | (33.3%) | 4 | (50.0%) | 6 | (42.9%) | |
| Fistula Size | | | | | | | 0.5105 |
| 1 mm | 2 | (33.3%) | 5 | (62.5%) | 7 | (50.0%) | |
| 2 mm | 2 | (33.3%) | 1 | (12.5%) | 3 | (21.4%) | |
| >6 mm | 2 | (33.3%) | 2 | (25.0%) | 4 | (28.6%) | |
| Fistula Etiology | | | | | | | 1.0000 |
| Inflammatory | 0 | (0.0%) | 1 | (12.5%) | 1 | (7.1%) | |
| Obstetric | 4 | (66.7%) | 4 | (50.0%) | 8 | (57.1%) | |
| Unknown | 0 | (0.0%) | 1 | (12.5%) | 1 | (7.1%) | |
| Postop/Trauma | 2 | (33.3%) | 2 | (25.0%) | 4 | (28.6%) | |
| Fistula Location | | | | | | | 1.0000 |
| Low | 6 | (100%) | 8 | (100%) | 14 | (100%) | |
| Mid | 0 | (0.0%) | 0 | (0.0%) | 0 | (0.0%) | |
| High | 0 | (0.0%) | 0 | (0.0%) | 0 | (0.0%) | |
| Prior Fistula Repair | | | | | | | 0.2774 |
| No | 4 | (66.7%) | 2 | (25.0%) | 6 | (42.9%) | |
| Yes (Mean no.) | 2 (1) | (33.3%) | 6 (3.2) | (75.0%) | 8 | (57.1%) | |

TABLE 2
Perioperative PROMIS* Measures

| | Total | Recurrent | Non-Recurrent | P-Value |
|---|---|---|---|---|
| Preop Physical Health Score | 48.3 (9.4) | 45.7 (10.4) | 52.5 (6.4) | 0.2222 |
| Preop Mental Health Score | 46.1 (6.7) | 44.5 (7.6) | 48.8 (4.0) | 0.2696 |
| Postop Physical Health Score | 46.2 (9.9) | 39.6 (6.5) | 54.0 (6.5) | 0.0058 |
| Postop Mental Health Score | 45.7 (10.0) | 40.1 (4.1) | 52.3 (11.2) | 0.0233 |

*Patient-Reported Outcomes Measurement Information System
T-scores are reported with standard error on T-score metric in parentheses
T-score distributions are standardized such that a 50 represents the average (mean) for the US general population, and the standard deviation around that mean is 10 points.

TABLE 3
Sample Distribution after Quality Control

| | T1 | T2 | T3 | T3R |
|---|---|---|---|---|
| Vaginal | 14 | 14 | 6 | 8 |
| Rectal | 14 | 13 | 5 | 8 |

T1: preoperative collection
T2: immediate post-op collection
T3: 6-8 week post-op collection
T3R: collection with fistula recurrence

TABLE 4
Omnibus p-values (indicating overall associations)

| | Vaginal | Rectal |
|---|---|---|
| Recurrence | 0.189 | 0.413 |
| Menopausal | 0.088 | 0.009 |
| BMIcat | 0.151 | 0.081 |
| Probiotic | 0.858 | 0.604 |
| Antibiotic | 0.400 | 0.341 |
| PriorGynSurg | 0.635 | 0.349 |
| VaginalInfect | 0.090 | 0.432 |
| PriorRepair | 0.867 | 0.928 |
| Vaginosis | 0.146 | 0.343 |

TABLE 5
Differentially abundant vaginal taxa at T1

| | P value* | Q value^{§} | Non-recurrence Mean | Recurrence Mean |
|---|---|---|---|---|
| Cyanobacteria | 0.022 | 0.149 | 0.00460 | 0.00002 |
| Tenericutes | 0.027 | 0.149 | 0.00389 | 0.00002 |
| Bacteroidetes; Bacteroidaceae | 0.009 | 0.088 | 0.13645 | 0.09526 |
| Bacteroidetes; Marinifilaceae | 0.006 | 0.088 | 0.00394 | 0.00168 |
| Bacteroidetes; Rikenellaceae | 0.001 | 0.039 | 0.01655 | 0.00544 |
| Firmicutes; Acidaminococcaceae | 0.017 | 0.095 | 0.02021 | 0.01325 |
| Firmicutes; Christensenellaceae | 0.007 | 0.088 | 0.00440 | 0.00062 |
| Firmicutes; Clostriadiales_vadinBB60_group | 0.013 | 0.095 | 0.00147 | 0.00000 |
| Firmicutes; Ruminococcaceae | 0.017 | 0.095 | 0.16739 | 0.07385 |
| Bacteroidetes; Alistipes | 0.02 | 0.060 | 0.01602 | 0.00531 |
| Bateroidetes; Bacteroides | 0.009 | 0.099 | 0.13645 | 0.09526 |
| Bacteroidetes; Butyricimonas | 0.012 | 0.104 | 0.00194 | 0.00000 |
| Bacteroidetes; Odoribacter | 0.032 | 0.190 | 0.00199 | 0.00168 |
| Bacteroidetes; Paraprevotella | 0.007 | 0.093 | 0.00539 | 0.00001 |
| Firmicutes; Christensenellaceae_R-7_group | 0.006 | 0.093 | 0.00396 | 0.00046 |
| Firmicutes; Dorea | 0.021 | 0.139 | 0.00197 | 0.00126 |
| Firmicutes; Faecalibacterium | 0.014 | 0.104 | 0.07313 | 0.01590 |
| Firmicutes; Family_XIII_AD3011_group | 0.019 | 0.133 | 0.00155 | 0.00070 |
| Firmicutes; GCA-900066225 | 0.028 | 0.175 | 0.00179 | 0.00018 |
| Firmicutes; Lachnospiraceae_ND3007_group | 0.01 | 0.099 | 0.00458 | 0.00007 |
| Firmicutes; Phascolarctobacterium | 0.014 | 0.104 | 0.01600 | 0.01125 |
| Firmicutes; Ruminococcaceae_NK4A214_group | 0.001 | 0.040 | 0.00281 | 0.00006 |
| Firmicutes; Ruminococcaceae_UCG-003 | 0.007 | 0.093 | 0.00158 | 0.00010 |
| Firmicutes; Ruminococcaceae_UCG-005 | 0.013 | 0.104 | 0.00307 | 0.00143 |
| Firmicutes; Ruminococcaceae_UCG-010 | 0.001 | 0.040 | 0.00100 | 0.00003 |
| Firmicutes; Ruminococcaceae_1 | 0.005 | 0.093 | 0.00392 | 0.00001 |
| Firmicutes; Ruminococcaceae_2 | 0.01 | 0.099 | 0.00984 | 0.00292 |
| Firmicutes; Subdoligranulum | 0.001 | 0.040 | 0.01271 | 0.00164 |
| Proteobacteria; Parasutterella | 0.004 | 0.093 | 0.00638 | 0.00012 |

*P value adjusted for BMI
^{§}Q value based on FDR of 0.2

TABLE 6
Differentially abundant rectal taxa at T1

| | P value* | Q value^{§} | Non-recurrence Mean | Recurrence Mean |
|---|---|---|---|---|
| Bacteroidetes; Rikenellaceae | 0.003 | 0.120 | 0.02122 | 0.00919 |
| Bacteroidetes; Alistipes | 0.002 | 0.083 | 0.01992 | 0.00880 |
| Bacteroidetes; Butryicimonas | 0.014 | 0.170 | 0.00141 | 0.00001 |
| Bacteroidetes; Paraprevotella | 0.017 | 0.177 | 0.00387 | 0.00004 |
| Firmicutes; Christensenellaceae_R-7_group | 0.01 | 0.170 | 0.00341 | 0.00088 |
| Firmicutes; Faecalibacterium | 0.004 | 0.100 | 0.08444 | 0.01741 |
| Firmicutes; Family_XIII_AD3011_group | 0.012 | 0.170 | 0.00190 | 0.00039 |
| Firmicutes; Phascolarctobacterium | 0.013 | 0.170 | 0.02109 | 0.01510 |
| Firmicutes; Ruminococcaceae_NK4A214_group | 0.004 | 0.100 | 0.00264 | 0.00016 |
| Firmicutes; Ruminococcaceae_UCG-003 | 0.005 | 0.104 | 0.00119 | 0.00011 |
| Firmicutes; Ruminococcaceae_UCG-010 | 0.001 | 0.063 | 0.00078 | 0.00004 |
| Firmicutes; Subdoligranulum | 0.001 | 0.063 | 0.01364 | 0.00125 |
| Firmui cutes; Succinispira | 0.015 | 0.170 | 0.00105 | 0.00022 |

*P value adjusted for BMI
^{§}Q value based on FDR of 0.2

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having a rectovaginal fistula (RVF), wherein said method comprises administering to said mammal a composition comprising one or more microbes selected from the group consisting of a Cyanobacteria species, a Tenericutes species, a Bacteroidetes species, a Firmicutes species, and a Proteobacteria species.

2. The method of claim 1, where the composition comprises one or more microbes selected from the group consisting of a Bacteroidaceae species, a Marinifilaceae species, a Rikenellaceae species, an Acidaminococcaceae species, a Christensenellaceae species, a Ruminococcaceae species, an Alistipes species, a Bacteroides species, a Butyricimonas species, an Odoribacter species, a Paraprevotella species, a Dorea species, a Faecalibacterium species, a Lachnospiraceae species, a Phascolarctobacterium species, a Subdoligranulum species, a Parasutterella species, and a Succinispira species.

3. The method of claim 1, wherein said mammal is a human.

4. The method of claim 1, comprising administering said composition to said mammal prior to surgical repair of said RVF.

5. The method of claim 1, further comprising identifying said mammal as having a rectal or vaginal microbiome with reduced levels of said one or more microbes, as compared to the rectal or vaginal microbiome in a corresponding mammal that does not have a RVF.

6. The method of claim 1, wherein said composition comprises from about 10³ cfu to about 10¹⁵ cfu of each microbe.

7. The method of claim 1, wherein said composition is administered orally, vaginally, or rectally.

8. The method of claim 1, wherein said composition is formulated as a tablet, a capsule, a pill, a nutritional supplement, or a beverage.

9. The method of claim 1, wherein said composition further comprises one or more prebiotics.

10. The method of claim 1, wherein said administering comprises fecal microbiota transplantation.

11. A method for altering the rectal and/or vaginal microbiome in a mammal, wherein said method comprises administering to said mammal a composition comprising one or more microbes selected from the group consisting of a Cyanobacteria species, a Tenericutes species, a Bacteroidetes species, a Firmicutes species, and a Proteobacteria species.

12. The method of claim 11, where the composition comprises one or more microbes selected from the group consisting of a Bacteroidaceae species, a Marinifilaceae species, a Rikenellaceae species, an Acidaminococcaceae species, a Christensenellaceae species, a Ruminococcaceae species, an Alistipes species, a Bacteroides species, a Butyricimonas species, an Odoribacter species, a Paraprevotella species, a Dorea species, a Faecalibacterium species, a Lachnospiraceae species, a Phascolarctobacterium species, a Subdoligranulum species, a Parasutterella species, and a Succinispira species.

13. The method of claim 11, wherein said mammal is a human.

14. The method of claim 11, wherein said mammal is identified as having a RVF.

15. The method of claim 14, wherein said method comprises administering said composition to said mammal prior to surgical repair of said RVF.

16. The method of claim 11, further comprising identifying said mammal as having a rectal or vaginal microbiome with reduced levels of said one or more microbes, as compared to the rectal or vaginal microbiome in a corresponding mammal that does not have a RVF.

17. The method of claim 11, wherein said composition comprises from about 10³ cfu to about 10¹⁵ cfu of each microbe.

18. The method of claim 11, wherein said composition is administered orally, vaginally, or rectally.

19. The method of claim 11, wherein said composition is formulated as a tablet, a capsule, a pill, a nutritional supplement, or a beverage.

20. The method of claim 11, wherein said composition further comprises one or more prebiotics.

21. The method of claim 11, wherein said administering comprises fecal microbiota transplantation.
